(19) United States
(12) Patent Application Publication
(10) Pub. No.: US 2016/0379741 A1
(43) Pub. Date: Dec. 29, 2016

(54) Low-Cost Precursor for Synthesis of High Coercivity Fe-N Magnets

(71) Applicant: Advanced Materials Corporation, Pittsburgh, PA (US)

(72) Inventors: Lawrence Frederick Allard, JR., Knoxville, TN (US); Michael P. Brady, Oak Ridge, TN (US); Craig A. Bridges, Knoxville, TN (US); Edgar Lara-Curzio, Lenoir City, TN (US); Ji Won Moon, Oak Ridge, TN (US); Orlando Rios, Knoxville, TN (US); Suryanarayan G. Sankar, Pittsburgh, PA (US); Brian Zande, Pittsburgh, PA (US)

(73) Assignee: Advanced Materials Corporation, Pittsburgh, PA (US)

(21) Appl. No.: 15/194,194

(22) Filed: Jun. 27, 2016

Related U.S. Application Data
(60) Provisional application No. 62/185,057, filed on Jun. 26, 2015.

(51) Int. Cl. H01F 1/11; H01F 1/06; C12P 3/00; H01F 1/053

(52) U.S. Cl. CPC H01F 1/11; H01F 1/053; H01F 1/06; C12P 3/00

(57) ABSTRACT

The disclosure describes a method of producing iron nitride magnets using Zn-doped iron oxide precursors. The iron oxide precursors are reduced and nitrided to produce a powder containing iron nitride in the Fe₁₆N₂ phase. The inclusion of Zn in the iron oxide precursor enhances the magnetic properties of the iron nitride powder.

### CROSS-REFERENCE TO RELATED APPLICATIONS

This application claims the benefit under 35 U.S.C. §119 of Provisional Ser. No. 62/185,057, filed Jun. 26, 2015, which is incorporated herein by reference.

### STATEMENT REGARDING FEDERALLY SPONSORED RESEARCH

This invention was made with Government support under DE-AC05-00OR22725 and DE-AR00-000645 awarded by the U.S. Department of Energy. The Government has certain rights in this invention.

### BACKGROUND OF THE INVENTION

This invention relates generally to iron-based magnets. More specifically, the invention relates to precursors used in the synthesis of iron nitride (Fe—N) magnets.

Iron nitride magnets based on the Fe₁₆N₂ phase are of great interest as a magnetic material for applications ranging from data storage to electrical motors for vehicles, wind turbines, and other power generation equipment. This is because the component base elements, iron (Fe) and nitrogen (N), are inexpensive and widely available, in contrast to rare earth element based magnets which are costly and subject to supply availability risks. The Fe₁₆N₂ phase, which is the ordered version of Fe₈N, is widely reported to have the largest magnetization of any compound, but is also difficult to manufacture.

The reduction of Fe-based oxide and hydroxide nanopowder precursors and conversion to Fe₁₆N₂ by nitridation is generally known in the art. See, for example, Sankar et al. (US 2011/0059005) or Ogawa et al. (EP 2 492 927). Sankar discloses one method of Fe₁₆N₂ manufacture where an iron oxide starting material (or precursor) is reduced in a fluidized bed reactor with a reducing agent, such as H₂, in a temperature range of 200-500° C. Subsequent nitridation of the reduced iron occurs through exposure in the fluidized bed reactor to pure NH₃ or NH₃—N₂—H₂ gases at 100-200° C. to form the Fe₁₆N₂ phase. Oxide coatings (e.g. alumina or silica) and/or metal dopants (Co, Cr, Mn, Ni, Ti, other transition metals, and rare earths) have been used to improve Fe₁₆N₂ phase yield, as well as improve resultant magnetic properties, such as magnetization and coercivity. While this method produces suitably high yields of Fe₁₆N₂, it has proven challenging to also achieve high levels of magnetization and coercivity, particularly when using sufficiently low cost iron-based precursor powders, which compose a major portion of the production costs.

As with the Sankar method and other methods, a key factor for a commercially viable Fe₁₆N₂ magnet is using low cost precursors that yield high quality Fe₁₆N₂, as the precursor cost dominates the cost of the resultant Fe₁₆N₂. Just as importantly, the size, consistency, and quality of the precursor ultimately affect the quality of the Fe—N magnet.

Potentially low cost processes for creating metal oxide nanoparticles have been previously developed by Oak Ridge National Laboratory (U.S. Pat. No. 6,444,453, Lauf et al., “Mixed oxide nanoparticles and method of making”; U.S. Pat. No. 7,060,473, Phelps et al., “Fermentative process for making inorganic nanoparticles”; and US Pub. 2010/0184179, Rondinone et al., “Microbial-mediated method for metal oxide nanoparticle formation”). However, the nanoparticles produced by the fermentative processes described in these references are not limited to iron oxides and have not been optimized for subsequent nitridation and incorporation into Fe₁₆N₂ magnets.

It would therefore be advantageous to develop an improved process for the synthesis of Fe₁₆N₂ nanopowders using low-cost precursors, where the nanopowders exhibit improved suitability for use in Fe—N magnets.

### BRIEF SUMMARY OF THE INVENTION

The present invention is directed to the use of doped bacteria fermented Fe-oxide nanoparticles as an improved, low-cost precursor for synthesis of Fe₁₆N₂ phase compounds.

### BRIEF DESCRIPTION OF THE SEVERAL VIEWS OF THE DRAWINGS

FIGS. 1A-1C are TEM images of undoped and Co- and Mn-doped Fe₃O₄ precursor powders, according to one embodiment, synthesized by bacteria fermentation.

FIGS. 2A-2C are TEM images of Zn-doped Fe₃O₄ precursor powders, according to one embodiment, synthesized by bacteria fermentation.

FIG. 3 is a graph of VSM magnetic hysteresis for a Zn-doped Fe₃O₄ precursor powder before and after reduction and nitridation treatment.

FIG. 4 is a TEM image of the Zn-doped Fe₃O₄ precursor powder analyzed in FIG. 3 after reduction and nitridation treatment.

### DETAILED DESCRIPTION OF THE INVENTION

Bacteria-fermentation derived Fe-oxide nanoparticles show good potential to form Fe₁₆N₂ using conventional reduction and nitridation approaches. In one embodiment, low-cost Zn-doped Fe oxide precursors 101 yield high coercivity (>1500 Oe) Fe₁₆N₂ containing powder 200 comprised of a plurality of nanosized particles 201. The high coercivity is unexpected because Zn is diamagnetic and has not previously been considered a candidate dopant 102 to enhance Fe₁₆N₂ formation. A further advantage of a Zn-doped precursor 101 is that the Zn helps protect the resultant Fe₁₆N₂ containing powders 200 from corrosion, both when stored prior to consolidation in bulk form and once consolidated.

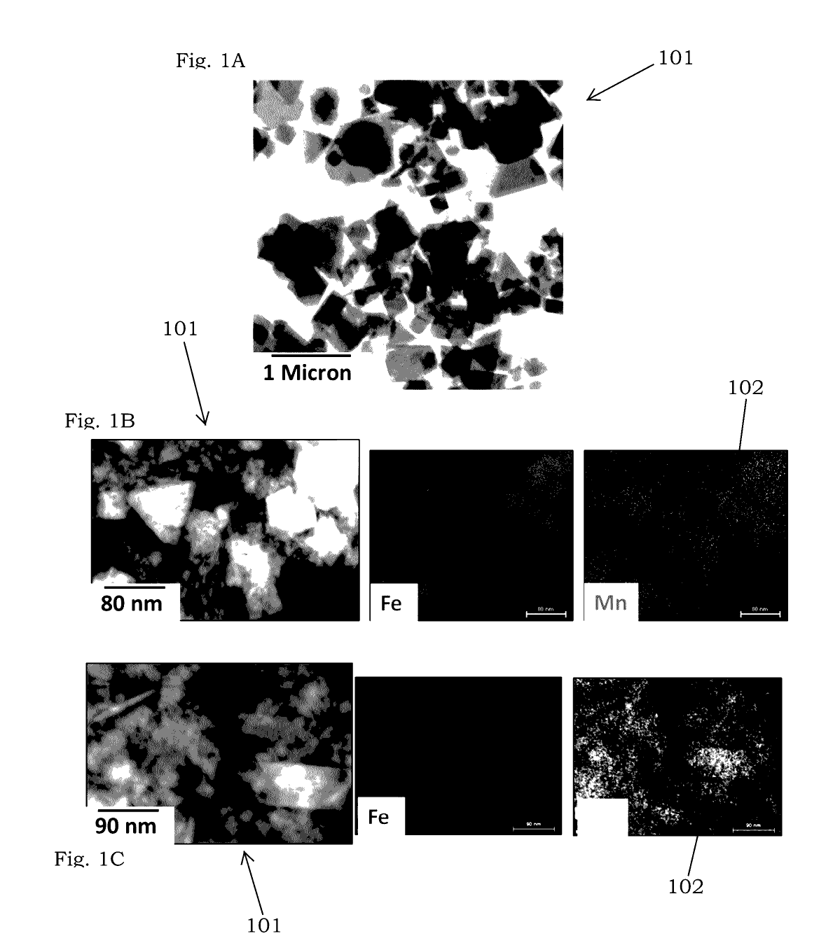

Transmission electron microscopy (TEM) images of bacteria-fermented Fe₃O₄ precursors 101, synthesized according to one method known in the art, are shown in FIGS. 1A-1C. FIG. 1A shows undoped Fe₃O₄ precursors 101, which consists of relatively large, nonuniform particles 201 having a diameter of about 0.1 to 1 microns. FIG. 1B and FIG. 1C are images of the precursor 101 doped with Mn and Co, respectively, with the dopants 102 well distributed throughout the precursor 101. In both FIGS. 1B and 1C, doping the precursor 101 resulted in a finer particle size, generally in the 50-100 nm range.

Despite their smaller particle size, initial reduction/nitridation trials using Mn- and Co-doped precursors 101 resulted in relatively low phase yields of about 12-30% Fe₁₆N₂. Similarly, coercivity values were in the 400-800 Oe range (see Table 1). These levels are unsuitable for viable commercial production of Fe₁₆N₂ powders 200. Further optimization of reduction and nitridation conditions can certainly be identified to increase Fe₁₆N₂ phase yield to more viable levels of 60-95% (with the possible exception of the 10 at. % Co-doped US-12 batch precursor 101, which exhibits a moderately attractive combination of coercivity over 780 Oe and magnetization over 210 emu/gram), but the screenings do not indicate sufficiently promising magnetic properties from these precursors 101 to warrant further development work with these dopants alone.

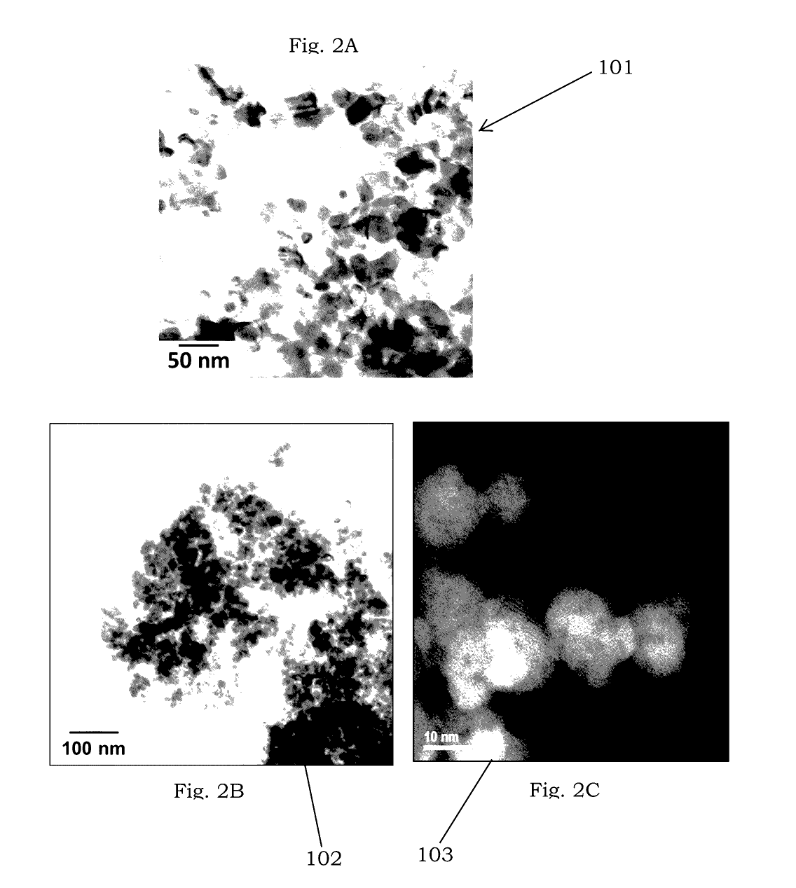

FIGS. 2A-2B show TEM images for several Zn-doped Fe₃O₄ precursors 101 synthesized using bacterial fermentation, with FIGS. 2B-2C showing precursors 101 with Zn substituted for Fe at the 1-10 at. % range. Zn dopants 102 in precursors 101 have not previously been utilized to enhance the magnetic properties of Fe₁₆N₂ containing ferromagnetic materials, unlike dopants 102 such as Mn and Co which exhibit paramagnetic/ferromagnetic behavior, because Zn is diamagnetic. However, the use of Zn dopants 102 in the bacteria fermentation process results in a very fine and uniform Fe₃O₄ precursor particle size, typically in the range of 10-40 nm and frequently centered in the 15-25 nm range.

Smaller particle size, which aids more rapid and uniform nitriding, and the presence of Zn can improve the qualities of the Fe₁₆N₂ powder 200 synthesized from the precursor 101. For example, the inclusion of Zn increases the coercivity of the final Fe₁₆N₂ product and is attributed, in part, to the fine, uniform, nanoscale precursor size imparted for Fe₃O₄ when doped with Zn. More specifically, the presence of Zn reduces sintering during reduction and nitridation due to a Zn-rich surface on the particles and Zn modifies the inter-/intra-particle magnetic interactions of the resultant Fe₁₆N₂. The incorporation of Zn/Zn oxide into the final product may also induce defects which favor increased levels of coercivity.

As shown in Table 1, unlike the precursors doped with Mn and Co, the Zn-doped Fe₃O₄ precursors 101 resulted in high Fe₁₆N₂ coercivity values, ranging from about 1100-1550 Oe. In the examples shown in Table 1, Zn is present in the precursor 101 in a range of 1-10 atomic percent (substitution of Zn for Fe). However, Zn can be present in the precursor 101 at different percentages if the resultant Fe₁₆N₂ powder 200 maintains acceptable magnetic properties. As such, a person having skill in the art will appreciate that the percentage of Zn can be adjusted based on the intended application of the Fe₁₆N₂ powder 200. Although reduction and nitridation of the Zn-doped Fe₃O₄ precursors 101 produced relatively low yield Fe₁₆N₂ powder 200 (about 8-40%) in initial trials, Zn-doped Fe₃O₄ precursors 101 permit the creation of Fe₁₆N₂ powders 200 with excellent magnetic properties, particularly high coercivity.

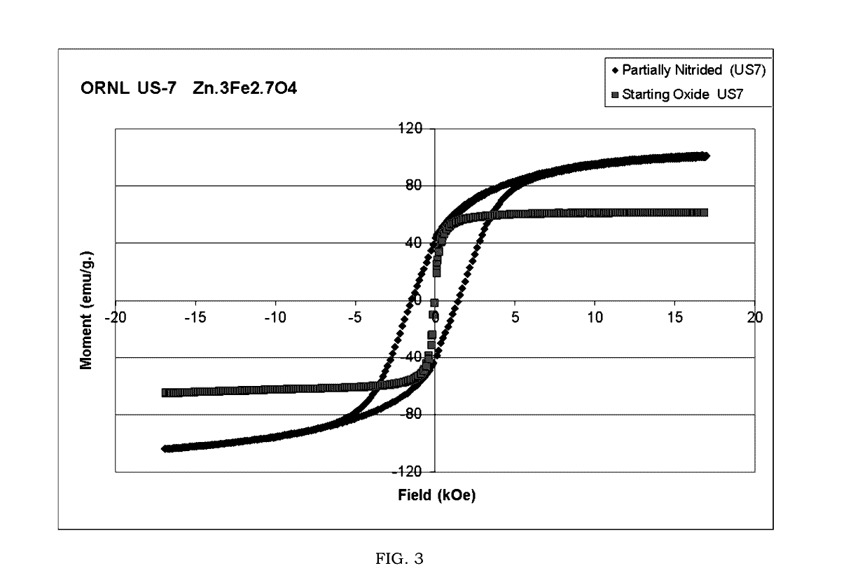

FIG. 3 shows magnetic hysteresis curves from a vibrating sample magnetometer (VSM) study of a 10 atomic percent Zn-doped Fe₃O₄ precursor 101, shown before and after reduction and nitridation. The coercivity of the untreated 10 atomic percent Zn-doped Fe₃O₄ precursor 101 is near zero, but reaches about 1500 Oe after reduction and nitridation. The high coercivity is despite only a ˜40% Fe₁₆N₂ phase yield. As indicated in Table 1, the measurements are conducted at ambient temperature (e.g. about 15-25 degrees Celcius).

An X-ray diffraction analysis of the resultant powder 200 indicates 41% Fe₁₆N₂, 17% Fe metal, and 42% incompletely reduced Zn—Fe—O. Unlike the powder 200 analyzed in FIG. 3, in the preferred embodiment, the Fe₁₆N₂ yield is about 60-95%, which can be accomplished through adjustment and optimization of the reduction and nitridation process time and temperature conditions to achieve more complete reduction of the Zn-doped Fe-oxide precursor 101 prior to nitridation. Such process optimization is widely reported in the literature via reduction/nitridation treatments using more conventional (non Zn-doped) types of Fe₃O₄ precursors 101. For example, in one process reduction occurs in H₂ in a temperature range of 200−500° C. and subsequent nitridation is accomplished through exposure in a fluidized bed reactor to pure NH₃ or NH₃—N₂—H₂ gases at 100-200° C. to yield high percentages of the Fe₁₆N₂ phase.

With optimization, in the preferred embodiment a Fe₁₆N₂ powder 200 created from low-cost Zn-doped precursors 101 has a coercivity level of about 2000-3000 Oe and magnetizations greater than 180 emu/g. Precursors 101 used in the preferred embodiment have Zn in the range of about 0.01 to 20 atomic percent substituted for Fe in the iron oxide precursor 101, with 1-10 atomic percent Zn preferred. In an alternative embodiment, co-doping of the precursor 101 can be performed to tailor and optimize magnetic properties with additions of Zn and at least one additional element from the group consisting of Al, B, C, Co, Cr, Hf, Mn, Nb, Ni, Si, Ta, Ti, V, Zr, and rare earths including Ce, La, Nd, Y, Dy, Sm at the 0.01-20 at. % level, with 1-10 at. % preferred.

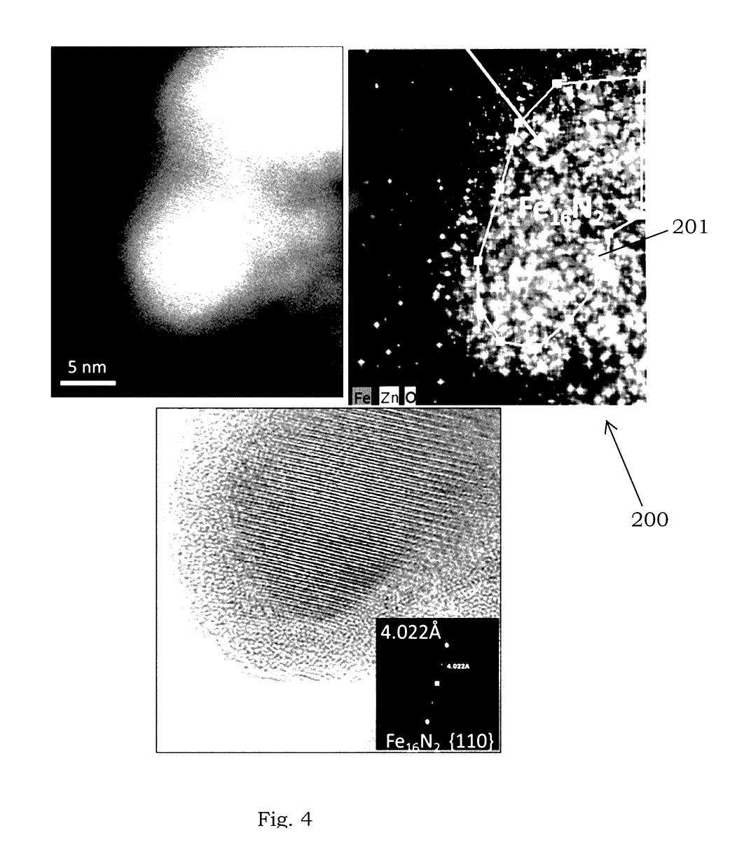

FIG. 4 shows high angle annular dark field TEM images of a Fe₁₆N₂ powder 200 synthesized from an iron oxide precursor 101 that was subjected to the reduction and nitridation process. The image in FIG. 4 confirms the formation of ordered Fe₁₆N₂. Elemental mapping suggests a core of Fe₁₆N₂, possibly surrounded by a Zn-containing oxide. That is, a coating 103 on the surface of the nanosized particle 201 contains one or more of the following: Zn, Zn—Fe—O, Zn—O, or Zn—Fe—N—O. From the image shown in FIG. 4, it is not possible to ascertain if any Zn was also incorporated directly into the Fe₁₆N₂ phase. The presence of intermixed Zn/Zn—Fe-0/Zn-0/Zn—Fe—N—O in the final product is anticipated to be beneficial from a stability and corrosion viewpoint, as Zn galvanization coatings are well established to protect steel from corrosion.

A high coercivity Fe₁₆N₂-containing powder 200 derived from low-cost Zn-doped bacteria fermented Fe₃O₄ precursors 101 with enhanced stability and corrosion resistance would be very attractive from a commercial scale processing approach standpoint, as well as for consolidation to bulk magnets. Limited stability and poor corrosion resistance considerations of current Fe₁₆N₂ powders 200 necessitate storage and consolidation strategies that minimize air exposure, and may result in higher production costs.

Table 1 is a summary of exploratory reduction and nitridation conversion reactions using bacterial fermented Fe₃O₄ precursors 101. Reduction is accomplished at 400-440° C. for up to 5 h in H₂, followed by nitridation in NH₃ at 160° C. and up to 20 h. The measured values are at ambient temperature (i.e. about 15-25 degrees Celcius).

| | Magnetic Properties after | | | Air Passivated Magnetic | |
|---|---|---|---|---|---|
| | nitridation, no passivation | | | Properties | |
| | Magnetization | Coercivity | | Magnetization | Coercivity |
| | at 16.5 kOe | H_{c} | Estimated % | at 16.5 kOe | H_{c} |
| Precursor | (emu/g) | (Oe) | Fe₁₆N₂ | (emu/g) | (Oe) |
| Undoped | 183.8 | 395 | NA | NA | NA |
| Fe₃O₄ | | | | | |
| Undoped | 193.3 | 504 | NA | 168.4 | 463 |
| Fe₃O₄ | | | | | |
| 20 at % Mn | 123.8 | 621 | 29 | 104 | 660 |
| doped Fe₃O₄ | | | | | |
| [US-5] | | | | | |
| 10 at % Co | 212.2 | 485 | 12 | 195 | 499 |
| doped Fe₃O₄ | | | | | |
| [US-47] | | | | | |
| 10 at % Co | 212.3 | 789 | 23 | 181.5 | 824 |
| doped Fe₃O₄ | | | | | |
| [US-12] | | | | | |
| 10 at % Zn | 100.8 | 1459 | 41 | 88.3 | 1548 |
| doped Fe₃O₄ | | | | | |
| [US-7] | | | | | |
| 10 at % Zn | 104.8 | 836 | 46 | 92.2 | 838 |
| doped Fe₃O₄ | | | | | |
| [US-53] | | | | | |
| 1 at % Zn | 185.7 | 1092 | 13 | 135.4 | 1089 |
| Fe₃O₄ | | | | | |
| 1 at % Zn | 194.3 | 1094 | 21 | 155.4 | 1090 |
| Fe₃O₄ | | | | | |
| 5 at % Zn | 186.9 | 1029 | 14 | 150.1 | 1066 |
| Fe₃O₄ | | | | | |
| 10 at % Zn | 150.4 | 820 | 8 | 115.7 | 870 |
| Fe₃O₄ | | | | | |

While the disclosure has been described in detail and with reference to specific embodiments thereof, it will be apparent to one skilled in the art that various changes and modification can be made therein without departing from the spirit and scope of the embodiments. Thus, it is intended that the present disclosure cover the modifications and variations of this disclosure provided they come within the scope of the appended claims and their equivalents.

What is claimed is:

1. A ferromagnetic Fe—N material comprising:
a plurality of nanosize particles forming a powder comprising Fe, Zn, N, and O,
wherein at least 25 weight percent of the powder comprises iron nitride in a Fe₁₆N₂ phase,
wherein about 0.01 to 20 atomic percent of the powder comprises Zn.

2. The ferromagnetic Fe—N material of claim 1, wherein about 1 to 10 atomic percent of the powder comprises Zn.

3. The ferromagnetic Fe—N material of claim 1, wherein the plurality of nanosize particles have a diameter of about 10-40 nm.

4. The ferromagnetic Fe—N material of claim 1, wherein the plurality of nanosize particles have a diameter of about 15-25 nm.

5. The ferromagnetic Fe—N material of claim 1, further comprising:
a coating on a surface of at least one of the plurality of particles, wherein the coating contains Zn.

6. The ferromagnetic Fe—N material of claim 5, wherein the coating comprise Zn, Zn—O, Zn—Fe—O, or Zn—Fe—N—O.

7. The ferromagnetic Fe—N material of claim 1, wherein a magnetic coercivity of the material is at least 1500 Oe at a temperature of about 15 to 25 degrees Celcius.

8. The ferromagnetic Fe—N material of claim 1, wherein the powder further comprises at least one additional element selected from the group consisting of Al, B, C, Co, Cr, Hf, Mn, Nb, Ni, Si, Ta, Ti, V, Zr, Ce, La, Nd, Y, Dy, Sm, and rare earth elements,
wherein about 0.01 to 20 atomic percent of the powder comprises the additional element.

9. The ferromagnetic Fe—N material of claim 8, wherein about 0.01 to 10 atomic percent of the powder comprises the additional element.

10. The ferromagnetic Fe—N material of claim 1, wherein the powder is derived from a Zn-doped iron oxide precursor,
wherein the precursor comprises 0.01 to 20 atomic percent Zn substituted for Fe,
wherein the precursor is a bacteria fermented iron oxide.

11. The ferromagnetic Fe—N material of claim 10, wherein the precursor further comprises at least one additional element selected from the group consisting of Al, B, C, Co, Cr, Hf, Mn, Nb, Ni, Si, Ta, Ti, V, Zr, Ce, La, Nd, Y, Dy, Sm, and other rare earth element,
wherein 0.01 to 20 atomic percent of the precursor comprises the additional element.

12. The nanopowder of claim 1 consolidated into a bulk ferromagnet form suitable for use in motors or other devices.

13. A method of fabricating a ferromagnetic Fe—N material comprising:
creating a Zn-doped iron oxide precursor using a bacteria fermentation process, wherein the Zn-doped Fe oxide precursor comprises 0.01 to 20 atomic percent Zn;
reducing the Zn-doped iron oxide precursor in H₂ at a temperature of about 200-500° C.;
nitriding the reduced iron oxide precursor in NH₃ or a NH₃—N₂—H₂ mixture at a temperature of about 100-200° C., producing a ferromagnetic material having at least 25 weight percent iron nitride in a Fe₁₆N₂ phase.

14. The method of claim 13, wherein the Zn-doped iron oxide precursor further comprises at least one additional element selected from the group consisting of Al, B, C, Co, Cr, Hf, Mn, Nb, Ni, Si, Ta, Ti, V, Zr, Ce, La, Nd, Y, Dy, Sm, and rare earth elements,
wherein about 0.01 to 20 atomic percent of the iron oxide precursor comprises the additional element.

15. A product by the process of claim 13.
